(19) United States
(12) Patent Application Publication
(10) Pub. No.: US 2018/0147767 A1
(43) Pub. Date: May 31, 2018

(54) METHODS OF EXTRUDING MULTILAYER FIBERS

(71) Applicant: THE BOARD OF TRUSTEES OF UNIVERSITY OF ILLINOIS, Urbana, IL (US)

(72) Inventor: Joshua M. Grolman, Urbana, IL (US)

(21) Appl. No.: 15/567,539

(22) PCT Filed: Apr. 25, 2016
(86) PCT No.: PCT/US16/29211

§ 371 Date: Oct. 18, 2017

Related U.S. Application Data
(60) Provisional application No. 62/152,124, filed on Apr. 24, 2015.

(51) Int. Cl. B29C 47/06; B29C 47/08; B29C 47/00; A61L 27/52; A61L 27/54; A61L 27/20

(52) U.S. Cl. CPC B29C 47/065; B29C 47/0811; B29C 47/0021; B29C 47/0004; B29L 2031/7532; A61L 27/54; A61L 27/20; B29C 2947/926; B29K 2005/00; A61L 27/52

(57) ABSTRACT

The present disclosure is directed to tubular fibers and methods of making thereof. In some cases, the fibers may be made of a hydrogel, in some cases an alginate hydrogel. The tube may have a nonsolid inner layer and an outer layer surrounding the inner layer. At least one of the inner layer and the outer layer may contain cells. In some cases, the tubular fiber may be used to study intercellular interactions.

### RELATED APPLICATION

The present patent document claims the benefit of priority under 35 U.S.C. 119(e) to U.S. Provisional Patent Application No. 62/152,124, entitled “3D EXTRUSION OF SYNTHETIC TUMOR MICROENVIRONMENTS,” filed on Apr. 24, 2015, and hereby incorporated by reference in its entirety.

### STATEMENT REGARDING FEDERALLY SPONSORED RESEARCH OR DEVELOPMENT

This invention was made with government support under contract numbers R00CA153914 awarded by the National Institutes of Health. The government has certain rights in the invention.

### BACKGROUND

### Technical Field Text

Solid tumors house an assortment of complex and dynamically changing microenvironments in which signaling events between multiple cell types are known to play a critical role in tumor progression, invasion, and metastasis. It is desirable to accurately model these structures in vitro for basic studies and for drug screening; however, current systems fall short of mimicking the complex organization of cells and matrix in vivo.

Anti-cancer drugs are typically assayed on tumor cell lines grown on tissue culture plastic with efficacy measured by growth inhibition or cell death. However, tumor progression in vivo is mediated by dynamic microenvironments where spatiotemporal control of signaling between diverse cell populations is responsible for growth and dissemination. Metastasis of breast cancer, in particular, is partially regulated by a paracrine loop between tumor cells (TC) and macrophages (Mϕ) in the primary tumor. This interaction enhances the motility of both cells and primes the TC to intravasate into the bloodstream, thus playing a key initiating event in disease progression. This heterotypic cell interaction pair has been directly observed in vivo using intravital microscopy and in vitro using a variety of 2D and 3D culture platforms. The development of therapeutic regimens that target heterotypic interactions preceding metastasis is an emerging area for development in cancer therapy. However, there is a deficit of in vitro systems that generate reproducible tissue morphology for a quantitative assessment of heterotypic signaling suitable for therapeutic development.

Compared to traditional 2D culture in a petri dish, 3D culture allows more accurate replication of natural tissue and matrix organization. In vitro models developed for drug screening have demonstrated differences in cell proliferation, morphology, and drug response for 3D compared to 2D systems. Microfluidic devices provide a means to organize 3D microenvironments such as cysts and tubules, which mimic the basic building blocks of epithelial tissue and allow high-surface-area interfaces between chemically or biologically distinct domains of tissue. Methods known for making certain media include a process in which chemical composition and topography are varied as a fiber is extruded, and a hydrodynamically-focusing method for generating cell-encapsulated fibers on a large scale has been developed. However, single channel fibers are limited in geometry, and rely on post-processing methods to achieve geometric variability and structural control.

It has been a challenge to develop a multilayer medium for maintenance and growth of cultured cells in which intracellular interactions can be observed and measured, particularly in a context in which the structures in which the cells are grown mimic natural vasculature.

### BRIEF SUMMARY

In one embodiment, a method of making a tubular fiber is provided. The method includes injecting a first fluid into a first flow channel at a first flow rate; injecting a second fluid into a second flow channel at a second flow rate, the second flow channel radially surrounding the first flow channel; and injecting a third fluid into a third flow channel at a third flow rate. The third flow channel radially surrounds the second flow channel and has an outlet downstream of the first and second flow channels. The method includes extruding a multilayer fiber from the outlet, the multilayer fiber having an outer layer comprising a hydrogel formed by gelation of the second fluid and an inner layer comprising the first fluid.

In another embodiment, a biological tissue model is described. The biological tissue model includes a multilayer fiber which has an inner layer including a nonsolid medium, and an outer layer surrounding the inner layer, the outer layer comprising a hydrogel.

In another embodiment, a kit for investigating intracellular interaction is disclosed. The kit includes a multi-well plate comprising a plurality of wells, each well containing a multilayer fiber segment. The fiber includes an inner layer which includes a nonsolid medium, and an outer layer surrounding the inner layer, the outer layer comprising a hydrogel.

### BRIEF DESCRIPTION OF THE DRAWINGS

FIG. 1 is a schematic of dual-cultured hydrogel fiber production in accordance with one embodiment of the present disclosure;

FIG. 2 is a photographic (a-b) and schematic (c-e) view of a device for generating fibers according to certain embodiments of the present disclosure;

FIG. 3 is a characterization and analysis of fiber structure in graphical (a), photographic (b-c), and scanning electron micrograph (e-i) form;

FIG. 4 is an NMR spectrum of alginate conjugated with YIGSR peptide (a-b) and chemical structures of alginate (c) and YIGSR-conjugated alginate (d);

FIG. 5 is a graphical representation of a physical study of fibers made according to a method of the present disclosure;

FIG. 6 illustrates the results of experiments in which fibers according to the principles of the present disclosure are used in a cell migration experiment;

FIG. 7 Illustrates the results of experiments in which fibers according to the principles of the present disclosure are used to gauge the influence of geometry on macrophage-tumor cell signaling;

FIG. 8 are photographs of stained cells which illustrate the principle of calculating a correlation factor of co-localization; and

FIG. 9 are photographs of stained cells which illustrate the principle of calculating cell number when using a fiber made according to the principles of the present disclosure.

### DETAILED DESCRIPTION OF THE DRAWINGS AND THE PRESENTLY PREFERRED EMBODIMENTS

Without wishing to be bound by any particular theory, there can be discussion herein of beliefs or understandings of underlying principles or mechanisms relating to embodiments of the disclosure. It is recognized that regardless of the ultimate correctness of any explanation or hypothesis, an embodiment of the disclosure can nonetheless be operative and useful.

The foregoing and other objects and features of the disclosure will become more apparent from the following detailed description, which proceeds with reference to the accompanying figures.

Further embodiments, forms, features, aspects, benefits, objects, and advantages of the present application shall become apparent from the detailed description and figures provided herewith.

The present disclosure demonstrates a versatile approach to multi-domain tissue mimetics by extruding multilayer fibers under controlled flow rates to modulate flapping instabilities that create folded, hierarchically conformed and/or overlapping fibrous masses representative of natural tissue. In order to mimic the structure of natural tissue and the interaction between various types of cells across the interface between layers, it is desirable to layer multiple substances in which cell cultures may be carried out one on another. One environment of particular interest is a tumor/blood vessel interface, which can be used for the study of tumor microenvironment (TMEM). In certain conditions in vivo, macrophages travel through blood vessels and respond at the site of a tumor, with such localization creating a proximity effect termed a microenvironment. Present three-dimensional cell culture models provide only a partial solution for creating an environment in which TMEM can be reproduced, but these lack the similarities to blood vessels and the surrounding tissue (that is, a solid or semi-solid matrix surrounding a vessel through which liquid can flow) that would best replicate biological reality. One such format for conducting such a study could be to form an elongate, multilayer fiber, with a more-solid outer layer, in some cases made at least in part of a hydrogel, surrounding a liquid-filled inner layer.

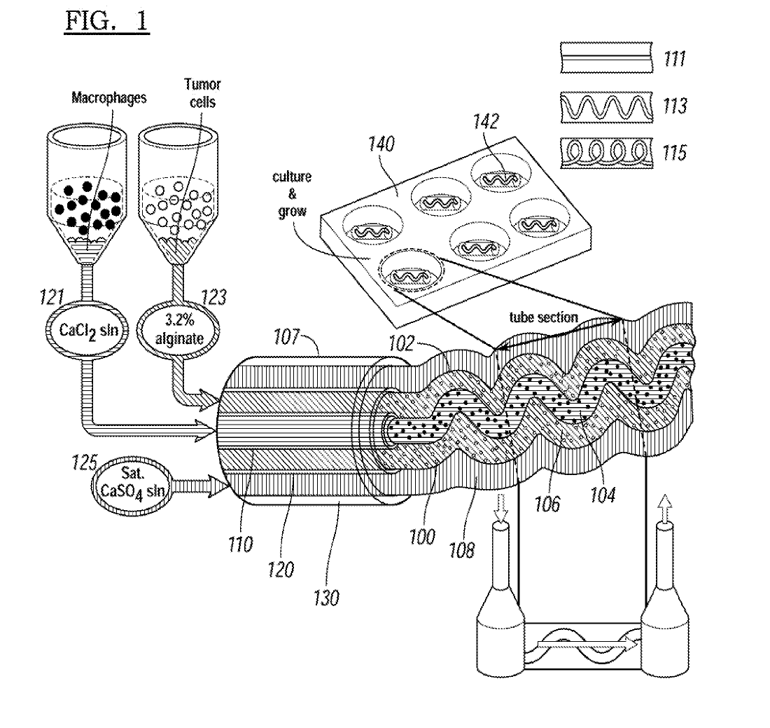

Referring to FIG. 1, a biological tissue model 100 comprises a multilayer fiber 102 including an inner layer 104 comprising a nonsolid medium, and an outer layer 106 surrounding the inner layer 104. The outer layer 106 comprises a hydrogel.

Referring to the upper right corner of FIG. 1, multilayer fiber 102 may have any of a number of morphologies, depending on the flow rates during extrusion, as discussed in detail below. For example, in one embodiment, the multilayer fiber 102 may have a straight structure 111, as shown in the topmost image. In yet another embodiment, the multilayer fiber 102 may not be a straight member, but may instead take on a curved shape, comprising at least one bend or curve. The precursor fluids which become the fiber may be flowed through the multichannel flow-focusing device such that the curved shapes are generated at a substantially regular frequency, giving rise to a serpentine structure 113, as shown in the middle image. In another embodiment, the multilayer fiber 102 may fold over on itself, with a first portion of the outer layer 106 coming into contact and overlying a second portion of the outer layer 106 in order to generate a folded structure, which may have a two-dimensional (2D) or three-dimensional (3D) “non-planar” type of architecture. One structure of this type may be a helical structure 115, as shown in the bottommost image, in which the fiber 102 forms a series of loops that are at least partially out of plane with one another. In some embodiments, the multilayer fiber may be extruded or arranged in such a 3D architecture to mimic the structure of various tissues, such as the vasculature of the breast, liver, colon, lung or any other tissue.

Advantageously, one or more cells may be included in the inner and/or outer layers 104,106. A single cell type or multiple cell types may be represented among the one or more cells. The cells may be viable cells, and may survive the process of fiber extrusion and formation such that their behavior within the fiber may be observed. The cells may be arranged such that a first type of cell or first cell line is included in the first fluid, and a second type of cell or second cell line is included in the second fluid. Alternatively, multiple types of cells or multiple cell lines may be included in the first fluid, the second fluid, or both.

In some embodiments, the hydrogel may comprise alginate. Controlling the fiber arrangement in a single fluidic extrusion step allows integration of multiple cell types in distinct and controllable spatial domains. This approach can allow for modeling tissue-mimetic interactions in vitro such as by, in one embodiment, filling the inner channel of the fiber with macrophages and incorporating tumor cells in the surrounding peptide-modified alginate. The 3D segregation of this cell pair can be observed over time, and pharmacological inhibitors of migration or TC-Mϕ signaling can disrupt normal spatiotemporal organization.

Spatially-organized 3D hydrogels of cells and matrix produced from a concentric flow device in a single step can be generated in one embodiment of the present invention. Multiple cell types can be pre-seeded in different spatial domains such as concentric regions of vessel-like tubular structures to reproducibly establish heterotypic cellular environments in three dimensions. In one embodiment, macrophages and breast adenocarcinoma cells may be employed as an example of a paracrine loop that regulates metastasis, enabling exploration of the effects of clinical drug treatments and observation of a dose-dependent modulation of cellular migration. This versatile and tunable approach for tissue fabrication represents a means to study a wide range of microenvironments and may provide a clinically-viable solution for personalized assessment of patient response to therapeutics.

One strategy for easily generating such fibers with high reproducibility is to simultaneously flow precursor solutions through a multichannel flow-focusing device 107 in a coextrusion method. For instance, to create a fiber 102 with a liquid (or nonsolid) center 104 and a solid outer layer 106 surrounding the inner, liquid layer 104, a first liquid 121 may be injected into the first (inner) flow channel 110 at a first flow rate, and a second liquid 123 which will later solidify may be injected into a second flow channel 120 which surrounds the first channel 110. The second liquid may have shear-thinning properties, and flowing a third liquid around this second liquid may assist in modulating the overall structure of the fiber which is formed. Such a third liquid may be flowed through a third flow channel which surrounds the second flow channel at a third flow rate. This third flow rate may be varied to adjust the structure of the fiber. The third flow channel extends beyond the end of the first and second flow channels so as to provide space for the more complex structures of the fiber to develop, if needed. In certain cases, the first fluid and the third fluid may include components that assist in gelating the second fluid, while the liquids containing such gelating agents remain nonsolid themselves.

Briefly, one exemplary strategy for making high-throughput co-cultured alginate fibers in a single step is illustrated in FIG. 1. First, human breast adenocarcinoma (MDA-MB-231; hereafter TC) cells and mouse macrophage (RAW 264.7; hereafter Mϕ) cells are optionally labeled with CellTracker and then mixed into pre-prepared about 3.2% weight alginate and about 0.046 g/mL CaCl₂ in Dulbecco's Modified Eagles' Media (DMEM) to form a second fluid 122 and a first fluid 120, respectively. The first fluid 121 is carried through a first flow channel 110, and the second fluid 123 is carried through a second flow channel 120 which radially surrounds the first flow channel 110. Optionally, a linker molecule for providing a further functionality to the eventual gel of the fiber may be provided. In one embodiment, such a linker may be a peptide, including in certain embodiments the pentapeptide sequence Tyr-Ile-Gly-Ser-Arg (YIGSR). This peptide can be conjugated by EDC/NHS to the alginate to support cell adhesion. A third fluid 125 is also present; in the illustrated case, this third fluid is a saturated solution of calcium sulfate, and the third fluid 125 is carried through a third flow channel 130 which radially surrounds the second flow channel 120.

Although the schematic illustration of FIG. 1 shows particular components, it will be apparent to one having skill in the art that many substitutions may be made and still fall under the scope of the present disclosure. For example, any tissue culture medium may substitute for DMEM in the first fluid, with the particular tissue culture medium selected to correspond with the type of first cell (if any) to be maintained in the inner layer 104 of the fiber that is generated. It is noted that the first fluid 121 as depicted in FIG. 1 also contains a first gelation agent. In this case the gelation agent is calcium chloride. However, depending on the downstream application and the desired characteristics of the fiber, a different first gelation agent can be selected. The first gelation may include a divalent cation, such as one chosen from among magnesium, barium, calcium, and other divalent cations, according to the speed at which gelation of the nascent fiber is desired. Other polymers beyond alginate may be selected for fiber materials, including for example MATRIGEL, agar, and agarose. The inclusion of cells in any of the first and second fluids is entirely optional and these may be excluded in certain cases.

In one embodiment, the multilayer fiber an inner layer which includes a nonsolid medium. The nonsolid medium in the example described above is a calcium-enriched DMEM, but may be any liquid, suspension, or nonsolid substance. The term “nonsolid” conveys that the inner layer does not itself solidify and does not become crosslinked. That is, a nonsolid portion is not a gel. The presence of a cell, or other particulate, in an environment that has the fluid characteristics of a liquid, will not be interpreted as thereby making the inner layer solid. A nonsolid inner layer 104 may be held in place by capillary action. The nonsolid inner layer 104 may have open ends such that another liquid might be flowed through the inner core of the fiber. In some cases, the ends of the fiber may be pinched together to close off the ends, but the nonsolid inner layer will still remain within the confines of the fiber.

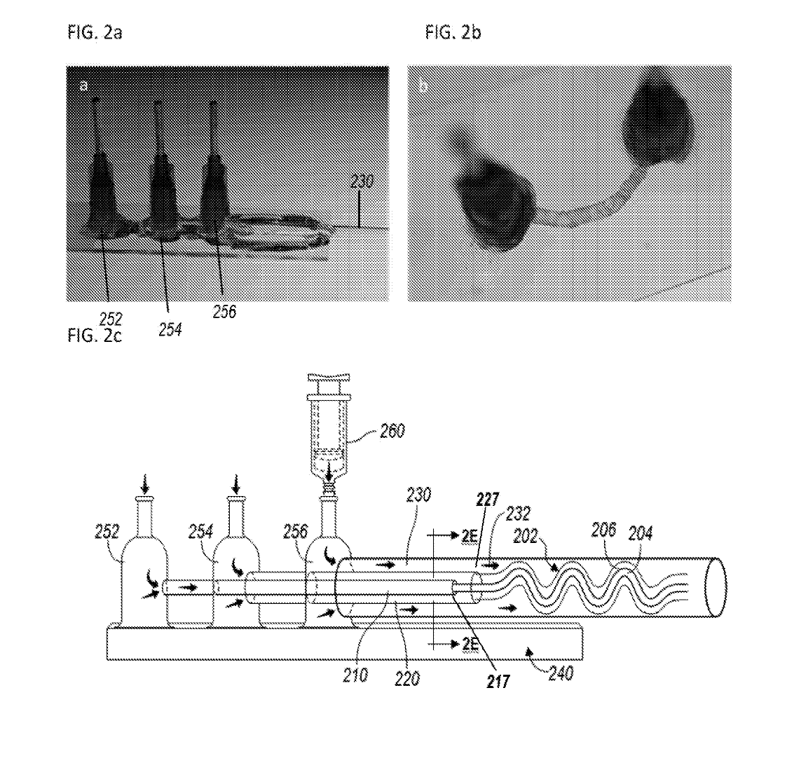

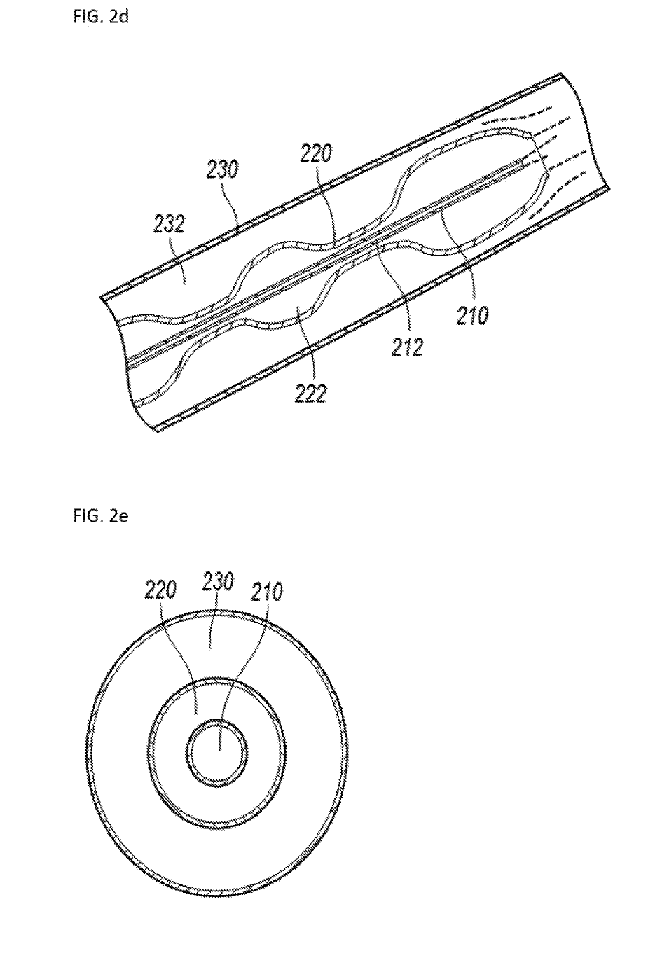

After being placed in an injection mechanism, in one embodiment syringe pumps, the solutions are extruded in the microfluidic device in the desired geometry as illustrated in FIG. 2, and collected in about 45 mg/mL CaCl₂ aqueous solution to aid in gelation of the polymer. As illustrated in FIG. 1, these fibers can then be cut into pieces 142 suitable for mounting as flowable tissue culture chips, or used in 96-well plates 140 for long term culture. In some embodiments, the fibers may be cut to lengths of about 1 centimeter (cm) to about 2 cm, as this sizing may assist in keeping the shape and higher-order architecture of the fiber. Such a length may be particularly well-suited for inclusion in the wells of standard microplates, as the fibers may fit into the wells by a friction fit. This makes changing the media simpler, as the fibers are less likely to move or become damaged since they remain relatively tightly held in the wells.

The flow-focusing device used to extrude the fibers may be assembled by placing glass capillary tubes with inner diameters of about 100 μm (microns), about 700 μm, and about 2000 μm in a concentric pattern, in some embodiments fixing them together, such as with glue. Each tube is connected to a corresponding inlet channel as shown in FIG. 2a. In certain embodiments, once a fiber has been extruded, it can be mounted in a similar manner and another fluid may be flowed through the inner channel, as shown in FIG. 2b.

A schematic cutaway side view of the device of FIG. 2a can be seen in FIG. 2c. In this embodiment, the structure of the flow-focusing device can be more clearly seen. The inlets 252/254/256 that correspond to flow channels are arranged from left to right in order of increasing flow channel diameter. Each of the flow channels 210/220/230 have a lumen for fluid to pass through, and in the cases of all but the smallest flow channel, for passage of the smaller flow channels. The first flow channel 210 passes through a chamber area of the second inlet 254, and is arranged coaxially within the second flow channel 220, running substantially parallel to it and occupying the longitudinal center of the second flow channel 220.

In some embodiments, and as illustrated in FIG. 2C, the outer (third) flow channel 230 may extend beyond the ends 217/227 of the first and second flow channels 210/220. This is because the first and second flow channels 210/220 contain substances that become the multilayer fiber 202, with the first flow channel 210 providing material for the inner (or core) layer 204, and the second flow channel 220 providing material for the outer (or shell) layer 206, whereas the third flow channel 230 is the outer environment in which the multilayer fiber takes shape, as described below.

FIG. 2d illustrates the motion of fluid through the various flow channels to create another fiber structure in accordance with another embodiment of the present disclosure. To generate this structure, a relatively greater quantity of gelation agent, such as calcium chloride, is included in the first fluid 212. This causes the middle fluid 222 to gel very quickly and produce a ‘straight vasculature’ structure, while Raleigh-Plateau instability causes the outside part of the second fluid 222 to form undulations within the context of the third fluid 232. These undulations later gel, resulting in a fiber with a repeating “pearl necklace”-like architecture. Such a structure may be useful in studying cellular interactions in environments where a vessel has, for example, a non-uniform thickness along its length.

FIG. 2e is a schematic view of the cross section of the flow-focusing device of FIG. 2c. Here, the relationship between the concentrically-aligned (e.g., coaxial) flow channels 210/220/230 can be observed. However, it may be difficult to achieve a perfectly concentric arrangement of the channels, so in some cases a substantially concentric arrangement or slightly coaxially off-set arrangement will be acceptable for operation of the flow-focusing device. Two flow channels will be said to have a “substantially concentric” arrangement if the inner channel does not contact the outer wall of the outer flow channel, such that the material of the outer channel can flow in such a way that material of the inner flow channel is completely surrounded by material from the outer flow channel in the extruded fiber.

It will be appreciated that throughout this application, the terms “extrude” and “extrusion” refer broadly to the movement of the multilayer fiber from out of the third flow channel. In some embodiments, the multilayer fiber begins to adopt its final shape (that is, straight, curved, folded, and the like) prior to extrusion. For example, and as shown in FIG. 2c, the multilayer fiber 202 is shown as adopting a sinuous structure within third flow channel 230 as it exits the second flow channel 220. In other embodiments, the multilayer fiber may not take on its final shape until after it exits the third flow channel. In some embodiments, the third flow channel opens into a bath or holding chamber for collection of fiber. Such a bath may be filled with a liquid, such as a gelating agent, tissue culture medium, or so forth, and may serve to allow the outer (or external) layer of the multilayer fiber to solidify. Extrusion may or may not involve contact by the fiber with the wall of the channel. The term “extrude” is used herein interchangeably with such terms as “expel” and “discharge,” among others.

Just as vertically extruded soft-serve ice cream twists into swirls when the end is seated in an ice-cream cone, hydrodynamically-focused alginate fibers are manipulated by an analogous push-back force that packs the extruded material into specific hierarchical conformations. Due to the shear-thinning nature of the alginate solution (second fluid) as it is extruded, the solution increases in its ability to bend, curve, rotate, or twist to accommodate for the stiffness exerted from the gelled, downstream second fluid. This viscoelastic characteristic allows for the formation of the overall structure of the fiber. Therefore, the second fluid may be considered a viscoelastic material. By running the second fluid at higher volumetric fluid flow rates compared to the third fluid, the second fluid tends to pack the extra volume by flapping back and forth in periodic arrangements.

The formation of the multilayer fibers as described herein differs from conventional 3D printing. Rather than relying on the motion of mobile print heads, it is the flow of fluid itself that works to shape the overall architecture of the tissue model and multilayer fiber. As a result, a relatively large quantity of fiber can be extruded over a given period of time, with high reproducibility and no loss of quality even as great lengths of fiber are rapidly produced. Moreover, in some embodiments an extended curing step may be avoided as the alginate hydrogel transitions from liquid to solid due to the presence of gelating agents in the first and third fluids. As opposed to structures generated by 3D printing methodologies, which in many cases can only take advantage of materials with specific intrinsic properties for consistent jetting, the method according to embodiments of the present disclosure has an increased ability to overcome differences in viscosity and surface charge variations of the materials used, thereby minimizing the effect from any deviations. Finally, a high degree of survival of cells that are incorporated into each layer of the multilayer fiber has been demonstrated, as seen below, which may not be a possibility with other methods of fiber formation.

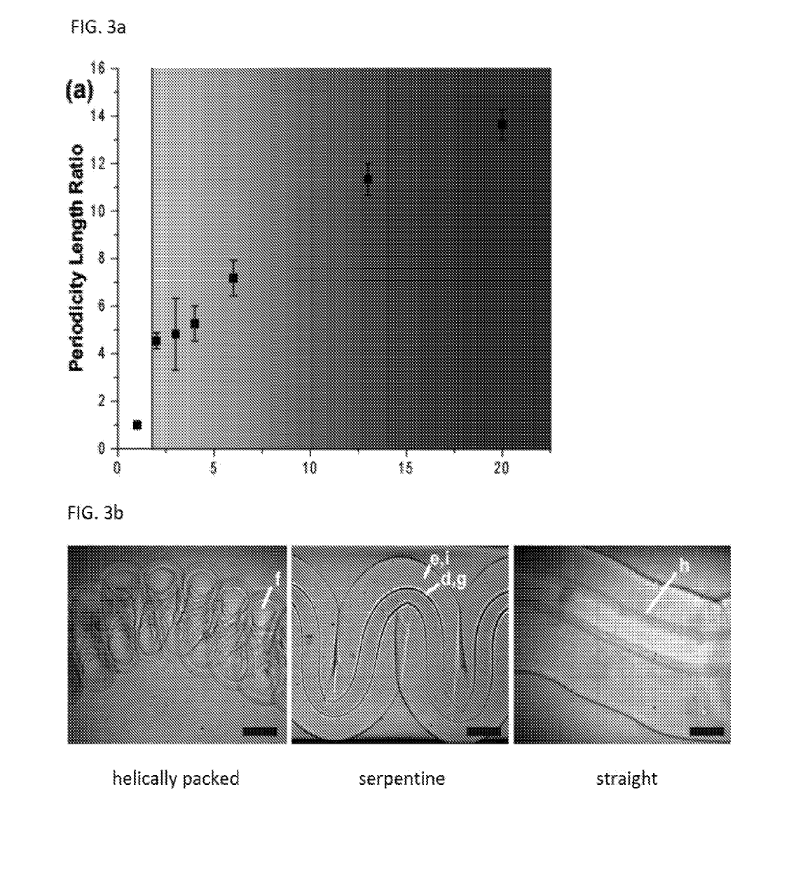

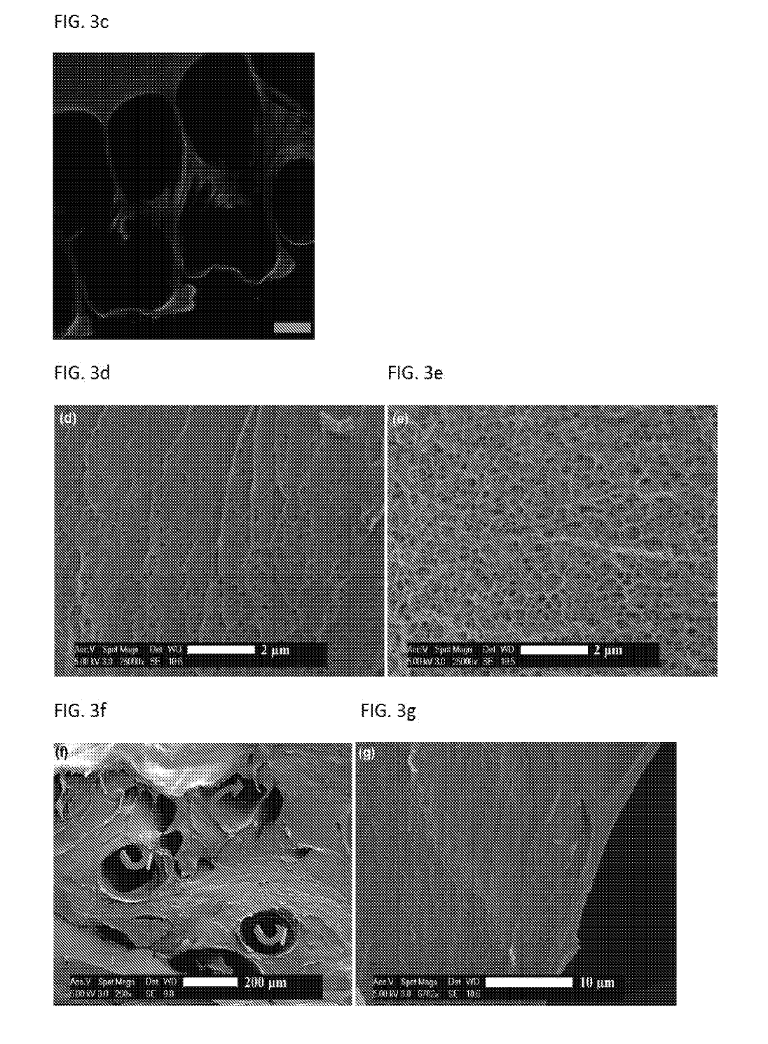

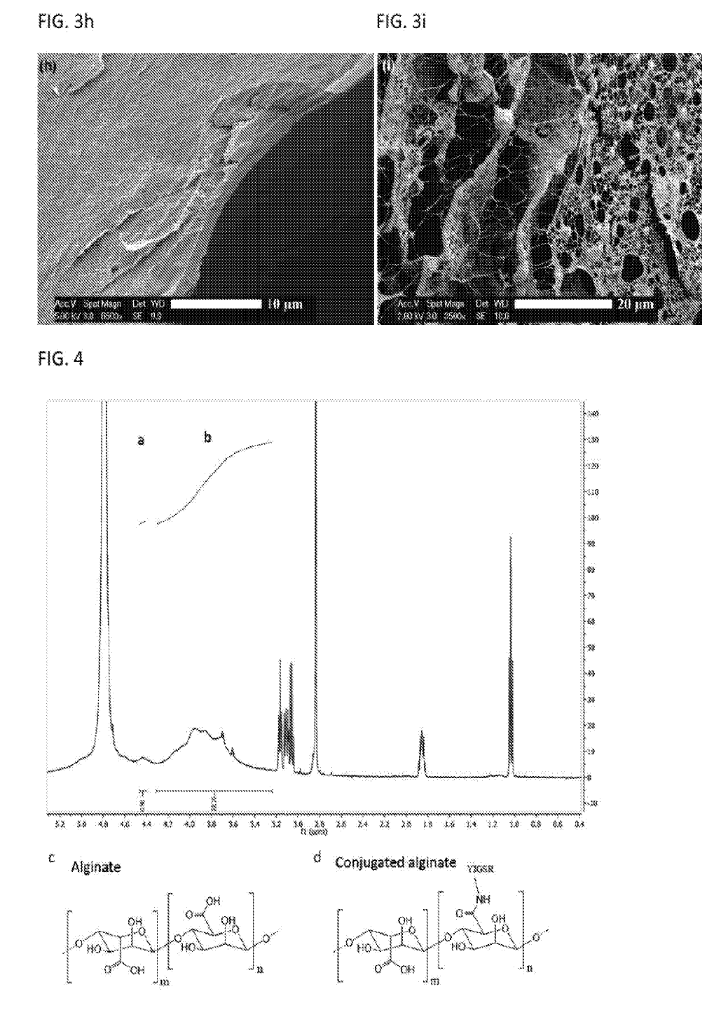

Based on results shown in FIG. 3a, decreases in the third fluid/second fluid volumetric flow rates result in tighter packing and form a single concentric co-flow device. FIG. 3a illustrates the architecture and pattern amplitude spacing dependence on third fluid/second fluid ratio. One-dimensional, two-dimensional, and three-dimensional architectures can be generated by changing the flow rate of at least one fluid relative to that of the other fluids being extruded, and thus the periodicity of the resulting serpentine fiber. However, this phenomena is observed at relatively high flow rates (second fluid=20 mL/hr). Using this strategy, the flapping frequency on-the-fly can be tuned to create hollow-channel fibers with multiple types of patterns on a continuously hollow calcium alginate hydrogel strand. Because sodium alginate is shear-thinning, a first gelating substance may be used as the outer (third) fluid (in one embodiment, saturated CaSO₄) to maintain temporal phase separation behavior until a second gelating substance, which may optionally be a different material from the first gelating substance (in one embodiment CaCl₂, at a concentration such as 45 mg/ml) in the inner channel diffuses radially throughout the fiber to ‘lock’ the structures into their respective architecture. In certain embodiments, the second gelating substance can cause more rapid gelation than the first gelating substance, as would be the case when calcium chloride is selected as the second gelating substance and saturated calcium sulfate is selected as the first gelating substance.

In an exemplary embodiment, a series of fibers can be generated by altering the flow rate through the various concentric, coaxial flow channels. For this exemplary embodiment, the first fluid has a first flow rate of about 2 milliliters per hour (mL/hr) through the first channel (center) having an internal diameter of 100 micron, the second fluid has a second flow rate of about 30 mL/hr through the second flow channel (middle) having an internal diameter of about 700 microns, and the third fluid has a third flow rate, which is varied, through the third flow channel (outer) having an internal diameter of 2000 micron. When the third flow rate is 100 mL/hr, the fiber that forms has a “straight” shape; that is, no three-dimensional hierarchy is realized, beyond the fact that the fiber appears to be a straight tubular member. When the third flow rate is slower, such as about 20 mL/hr, the nascent fiber takes on an undulating, periodic, serpentine form, with consistent peak/valley structure emerging in a predictable fashion. Slowing the third flow rate somewhat, to about 15 mL/hr, a serpentine form is still observed. Slowing the third flow rate yet further, to about 10 mL/hr, a new architecture emerges: this time, a helical fiber is formed as the slowing of the third fluid allows for the fiber to loop over unto itself. However, at about 10 mL/hr, such a structure is somewhat unstable, but if the third flow rate is slowed yet more, to about 5 mL/hr, a periodic, predictable, and stable helix of fiber can be generated.

The flow rates of each fluid can be varied, either individually, or with the flow rates of other fluids. In some cases, the second flow rate can be from about 12 mL/hr to about 30 mL/hr, inclusive, and any value between. In some embodiments, the third flow rate can be from about 2 mL/hr to about 200 mL/hr, inclusive, and any value between.

Without wishing to be bound by any theory, the ratio of the first, second, and third flow rates may drive the formation of the structures of nascent fibers. For instance, the first and second flow rates do not necessarily have to be 2 mL/hr and 30 mL/hr respectively, but may instead have a ratio of about 15, and may therefore function similarly. The flow rate ratio may be adjusted if the ratio of the internal diameters or cross-sectional areas of the center channel and the middle channel is different than the embodiment disclosed. A person having skill in the art will appreciate that properties of the various fluids, such as viscosity, charge, polar/nonpolar nature, and so forth, may also be taken into account to obtain different ratios of flow rates for optimal performance.

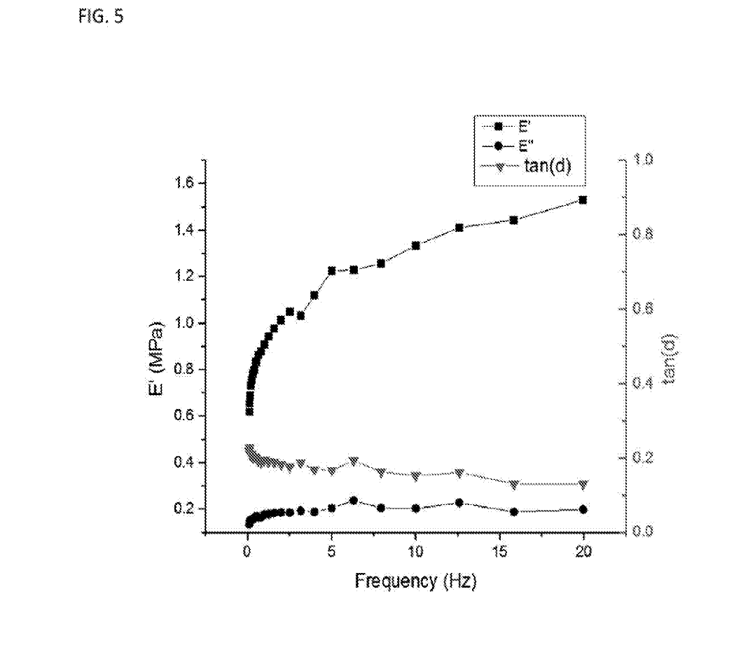

FIG. 3b shows optical images of select patterned fiber structures with 200 μm scale bars, and FIG. 3c is a confocal fluorescence image slice of covalently-conjugated fluorescein to a ‘3D’ fiber with a 50 μm scale bar. Geometric structures and porosity of the formed multilayer fibers, which may be considered as liquid-filled hollow channels may be observed by a combination of frame captures from high speed video recording (FIG. 3b), confocal fluorescence microscopy of the hydrogel covalently conjugated to fluorescein (FIG. 3c), dynamic mechanical analysis (FIG. 5), and environmental scanning electron microscopy (ESEM) (FIG. 3d-3i).

FIGS. 3d-3i illustrate calcium-alginate fibers prior to culturing with cells in accordance with one embodiment of the present invention. In the case of FIG. 3d, close-up ESEM image of tightly-cross-linked inner channel membrane indicating the smaller, partially collapsed pores of the calcium-alginate matrix, displaying characteristics of a hydrogel. In FIG. 3e, ESEM of the bulk aspect of the alginate fiber matrix is shown, and in FIG. 3f, a diagonal slice of a patterned alginate fiber showing intersecting inner channel segment is displayed. In FIG. 3g, ESEM of patterned fiber close to the opening of the inner channel (the core of the fiber). FIG. 3h is a macro image of straight fiber near inner channel opening, and FIG. 3i shows a Calcium-Alginate network freeze-dried after culturing with macrophages for four days (control media sample).

ESEM of the freeze-dried alginate demonstrates that pore size is homogenous within the bulk structure (FIG. 3e) with the exception of an approximately 2 micron crust of tightly-cross-linked hydrogel that surrounds every hollow inner channel (FIG. 3g-3h). In some embodiments, this crust may prove advantageous for applications where several levels of spatial cellular organization are desired (e.g. endothelial perfusion on the channel wall). Inner channel periodicity does not affect cross-linking or porosity in the bulk. Significant structural changes of the hydrogel fibers suggestive of remodeling are seen after 4 days culture with macrophages (FIG. 3i). This is evidence of both viability and of motility of the incorporated cells, in this case macrophages.

In certain embodiments, the polymer that makes up the hydrogel, outer layer of the multilayer fiber may be alginate. In another embodiment, the polymer may be modified to give the polymer an additional property. In one example, alginate may be conjugated to a peptide that promotes cell adhesion. In FIG. 4c, the structure of alginate is illustrated, and in FIG. 4d, a conjugated alginate which includes the cell-adhesion peptide Tyr-Ile-Gly-Ser-Arg (YIGSR) is shown. FIG. 4 further provides an NMR spectrum showing that YIGSR was incorporated into the alginate. Peak a correlates with the 4 backbone protons from YIGSR and peak b correlates with the manuronic and gluronic acid ring protons. Approximately 7% conjugation of peptide to the alginate backbone was realized in this example. A multilayer fiber can be made by utilizing this conjugated alginate according to the flow rate ratios disclosed herein.

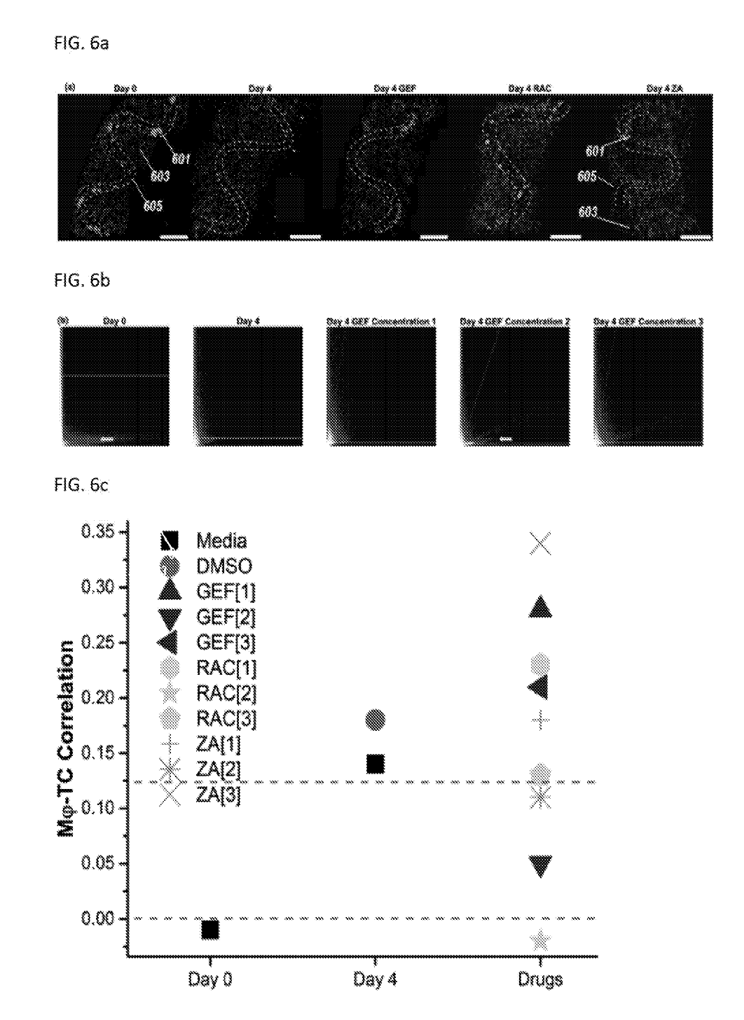

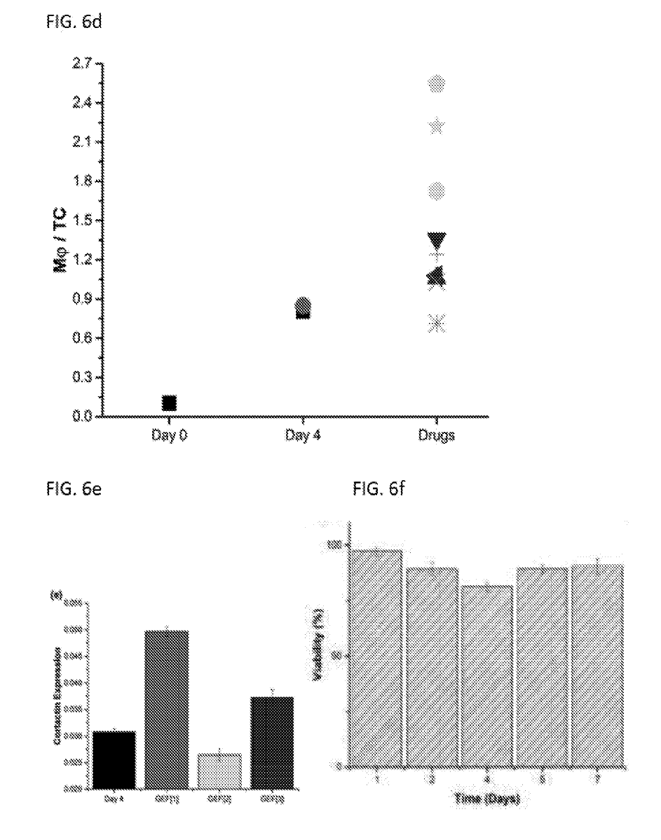

FIG. 6 demonstrates a co-localization and inhibition analysis of heterotypic co-cultures of macrophage-tumor cells. FIG. 6a presents 3-D reconstructions of macrophages (RAW) 601 surrounded by mammary tumor cells (231) 603 and stained with CellTracker, and the location of the nonsolid inner layer hollow inner channel indicated in dotted lines 605. In FIG. 6A, the scale bar is 400 μm.

FIG. 6c is a plot of the calculated Pearson Correlation factors for three drugs at three concentrations (at low[1], medium[2], or high[3] concentrations) in which over 90% of encapsulated cells were viable within the first week of culture (FIG. 6c; also FIG. 6f), with no significant changes in viability over 3 weeks. In some instances, tumor cells in vivo attract macrophages that secrete epidermal growth factor (EGF) to enhance the metastatic phenotype, thereby priming the tumor cells to intravasate into the vasculature. This paracrine interaction is proposed as a central event mediating metastasis. The multilayer fibers disclosed herein, including those formed from alginate hydrogels, provide a model system of TC-Mϕ co-culture for optimizing pharmacological compounds that disrupt this clinically relevant interaction. To illustrate this, co-culture supplemented with Gefitinib (GEF), an epidermal growth factor receptor (EGFR) inhibitor; zoledronic acid (ZA), a bisphosphonate that targets osteoclasts and macrophage cells; and a Rac1 inhibitor (RAC) as a broad spectrum modulator of cell migration were included in the model system to investigate motility of various cells within the fiber.

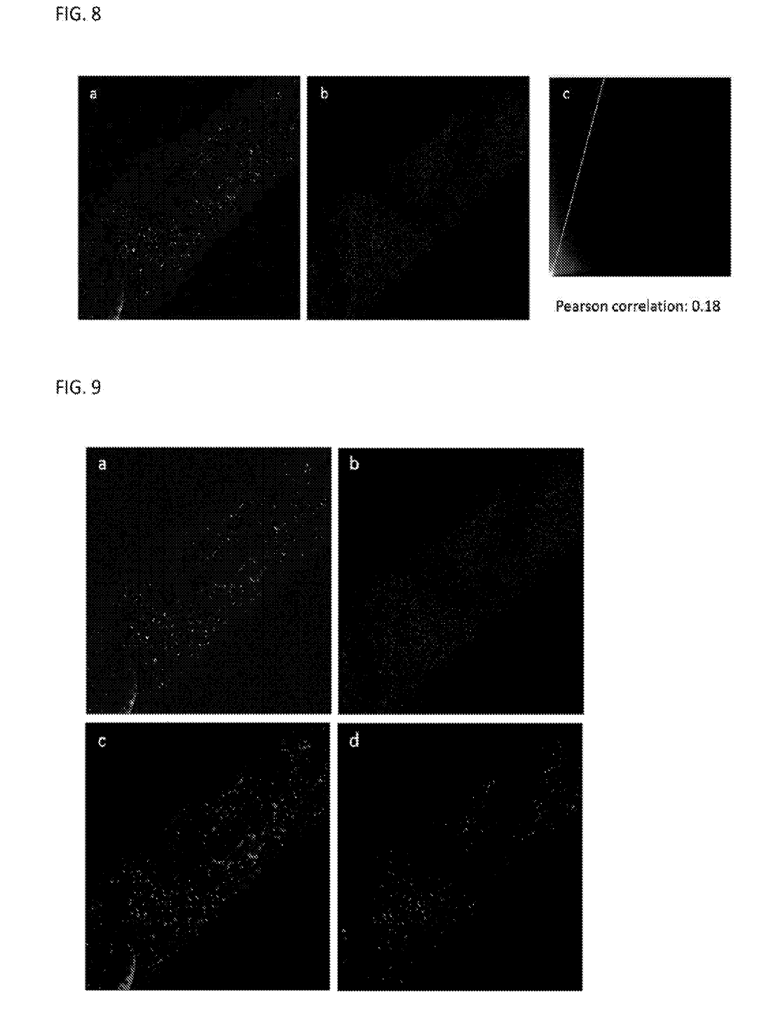

In FIG. 6b, co-localization scatter plots indicating a trend toward correlated pixel values between CellTracker fluorescent channels, which can be partially reversed upon incubation at certain drug concentrations is shown. Using CellTracker™-labeling and confocal fluorescence microscopy to quantify the co-localization of the tumor cells and macrophages in co-culture over time (FIG. 8). To calculate correlation factor, ImageJ Fiji with the Coloc2 plugin may be utilized. The images are split into separate channels for macrophages (a) and breast cancer (b) cells and despeckled to remove noise. The co-localization test may be run with threshold values of 12 for channel a, and 30 for channel b. The reported Pearson's R-value above the threshold is used for correlation factor.

At Day 0, the fiber samples are distinct and the macrophages are exclusively located in the hollow channels of the fibers (FIG. 6A, left panel). However, after four days of incubation in media or vehicle control, the macrophages became interspersed amongst the entire calcium-alginate hydrogel with a high degree of co-localization with tumor cells (FIG. 6a, second panel). When the co-cultures are treated with GEF, ZA, and RAC, there is a distinct impairment of migration and co-localization with the majority of macrophages remaining in the channel interior (FIG. 6a, right panels).

FIG. 6d illustrates a plot of the RAW cell to 231 cell volume ratio per fiber. When quantitating the co-localization of cell-specific CellTracker™ fluorescence signal using the Coloc2 plugin of ImageJ Fiji, at Day 0 when the two cell types are localized in distinct regions, there is a strong anti-correlated band indicated by the arrow in the fourth panel of FIG. 6b, which corresponds with a negative calculated correlation factor. After 4 days, when significant numbers of macrophages migrate within the alginate matrix, this anti-correlation band disappears, and is replaced by an increase in the calculated correlation factor (see FIGS. 6b and 6d). When cultures were treated with drug concentrations at the approximate IC₅₀ of all three inhibitors (50 μM for ZA and Rac1; 50 nM for GEF), the calculated correlation factor decreases. In particular, the inhibitor of Rac1, which is expected to impair cell migration between channels, leads to a return to anti-correlation comparable to initial seeding (Day 0). Supplementation of the cultures with drugs well below or above the IC₅₀ fails to abrogate the co-localization.

FIG. 6e illustrates cell-normalized cortactin expression from tumor cells over the course of three concentrations of GEF. There is an increase in the TC/Mϕ ratio from Day 0 to Day 4 in all of the samples, presumable because macrophage growth rates are nearly double that of the 231 tumor cells (FIG. 6e). Cells exposed to RAC show significantly higher TC/Mϕ ratios compared to the other samples, which may be due to continued proliferation in the channels upon motility inhibition. Not wishing to be bound by any particular theory, 231 cell death may also be a contributing factor.

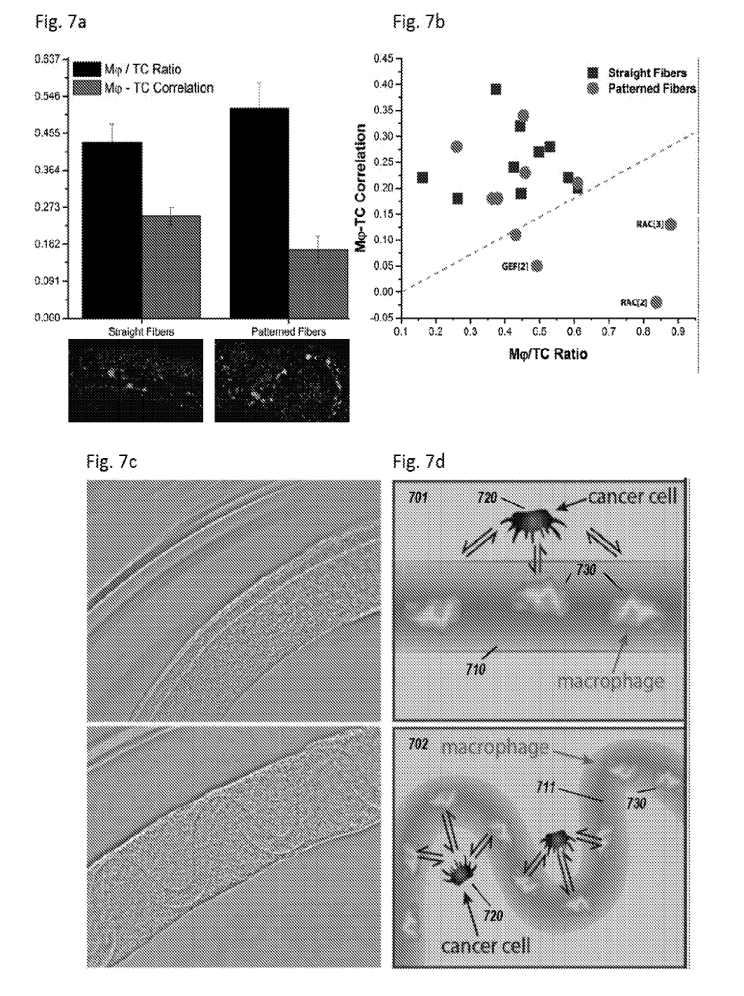

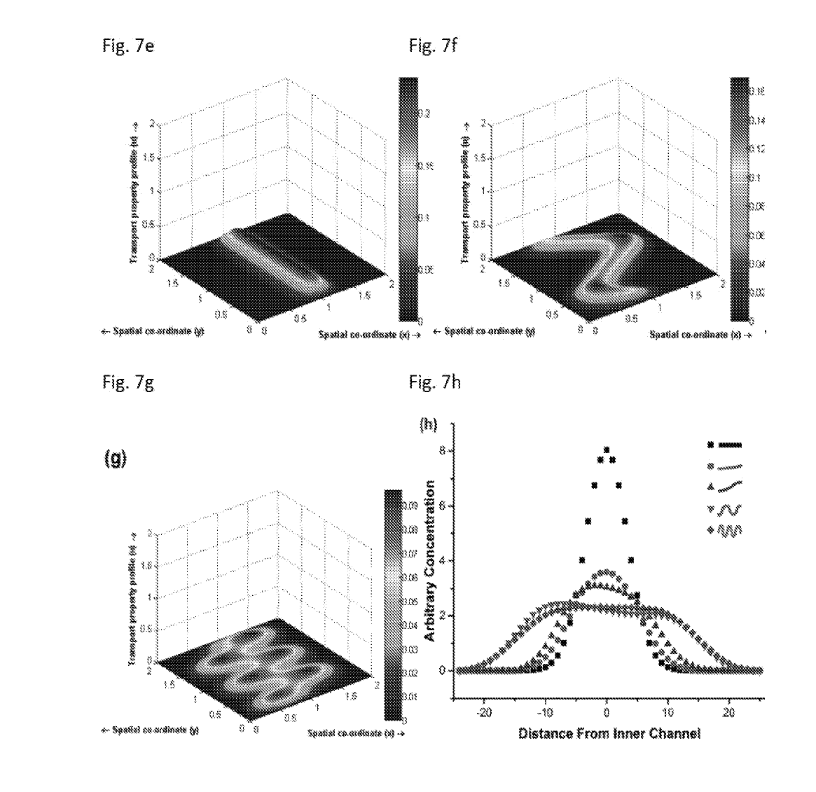

Turning now to FIG. 7, a comparison of cellular interaction experiments in fibers of differing architecture is presented. In FIG. 7a, a comparison of averaged macrophage (Mϕ))/tumor cell (TC) ratios to Mϕ-TC correlation factors is calculated for straight and patterned fibers respectively after drug and media exposure. In FIG. 7b, a plot relating the Mϕ-TC correlation with the Mϕ/TC ratio is displayed, with labels and a line drawn to illustrate the best performing conditions inhibiting macrophage migration. In FIG. 7c, straight and patterned hollow alginate structures formed according to the principles of the present disclosure are shown, with 200 μm scale bars. FIG. 7d is a cartoon illustration comparing how the geometric spatiality of cells may affect their signaling in naturally-occurring architectures and model systems. As shown in panel 701, a linear fiber 710 provides for an interface for interaction between tumor cells 720 and macrophages 730, but only allows for a close-proximity interaction between the cells of different types on a one-to-one basis, with macrophages 730 which are not directly across the interface from a tumor cell 720 having a limited influence on the tumor cell 720 (and vice versa) as signaling molecules have a longer path to traverse between the two different cells. Contrarily, in panel 702, the sinuous architecture 711 of the fiber allows for multiple macrophages 730 to influence a single cancer cell 720 at substantially equal distances, which may be a better model of vasculature in vivo. FIGS. 7e-7g show simulations of 2D anisotropic diffusion through Finite Difference Method for channels of e, zero; f, one; and g, two and a half periodic patterns.

By moving from the ‘1D’ fibers to the ‘2D’ patterns, the TC/Mϕ ratio increases. Comparing fibers with straight ‘1D’ channels to fibers with ‘2D’ wave-like architecture, the straight fiber samples consistently show higher correlation factors and thus more migration of macrophages to the alginate than their patterned counterparts (FIG. 7a). This trend is in contrast to the ratio of TC/Mϕ where the straight fibers result in a 50% decrease in the fraction of macrophages to tumor cells. This is presumably because of the lower volume of the inner channel in the ‘1 D’ compared to ‘2D’ fibers.

When comparing the TC-Mϕ correlation to the TC/Mϕ ratio for fibers treated with pharmacological inhibitors, drug treatments can be readily identified that show the highest inhibition of migration and co-localization (FIG. 7b; GEF and RAC). The different behavior of cells in the ‘1D’ fibers versus the ‘2D’ fibers may be on account of increased interactions, not only due to the increase in macrophages, but also from the directionality of the signaling (as illustrated in FIG. 7c). In living systems, structures can develop during normal morphogenesis and pathological processes to adopt a breadth of curvilinear and fractal-like forms (e.g. blood vessels, respiratory buds, mammary ducts), where diffusional distances and spatial positioning of cells is critical for function. The tissue-mimetic fibers according to the present disclosure may better emulate the non-linear architecture in living systems, indicating that this technique may find broad applicability in fabricating model tumor architectures for therapeutic development. FIG. 7h in particular shows the distributions of cellular distance from the inner layer of a multilayer fiber for different fiber architectures or structures.

In some embodiments, the multilayer fiber of the present invention may be provided to researchers in the form of a kit. The fiber may be generated from a flow-focusing device as described herein, cut to the appropriate size, and stored in either a calcium-containing gelation solution to solidify the fiber, or in a tissue culture medium for the growth and proliferation of cells. The kit may be manufactured to have a shelf life for storage, or may be manufactured as needed and on-demand so as to ensure freshness and stability of the fiber. The kit may be made with a preservative or a stabilizing agent, so that the fiber, the cells, the tissue culture medium, and so forth remain usable for a certain period after packaging. The kit may also include cells, drugs, growth factors, and any other components a researcher may need to maintain and study cells. In certain embodiments, the kit may provide cells expressing fluorescent proteins, such as green fluorescent protein, either by itself or as a fusion protein construct. The kit may be in a plate format, such as a 96-well plate in which a segment of fiber is placed in every well.

The kit may be supplied as a multi-well plate comprising a plurality of wells, each well containing a multilayer fiber segment comprising an inner layer comprising a nonsolid medium, and an outer layer surrounding the inner layer, the outer layer comprising a hydrogel. The kit may include a first type of cell in the inner layer and optionally at least one of a second type of cell in the outer layer. In a particular embodiment, the first type of cell may be a macrophage. In another embodiment, the second type of cell may be a cancer cell. In a specific embodiment, the first type of cell may be a macrophage, and the second type of cell may be a cancer cell. The outer layer may include alginate. In the kit, the hydrogel may further include a linker molecule. In some cases, the linker molecule in the kit may be a peptide. The kit may provide a multilayer fiber with a curved structure. The kit may provide a multilayer fiber having a serpentine structure. The kit may contain a stabilizing agent in some or in all wells of the plate.

Although the interaction between macrophages and cancer cells for study of tumor microenvironment has been demonstrated in this disclosure, a person of skill in the art will appreciate that interactions between many different pairs of cells, or more than two types of cells, can be studied using a model vasculature system according to the present invention.

The present disclosure provides an improvement over traditional microfluidic concentric flow spinning methods using fiber packing minimization to produce a variety of structures in a single simple device. The ability to quickly tune the packing of vascularized alginate multi-cell tissue scaffolds lends itself for use as a model system to study other heterotypic interactions. Indeed, this system may be particularly useful for modeling metastasis because the vessel architecture can be tuned on-the-fly. Not only are the scaffolds easily manufactured, they also offer tremendous potential as model systems for high-throughput screening of drug efficacy, as well as flow-able and vascularized lab-on-a-fiber platforms. This packing is not limited to, in certain cases, the gelation of calcium-alginate hydrogel fibers, but is applicable to a wide range of experimentation required for fast-patterning vasculature in the future.

### EXAMPLES

### Example 1

### Co-Culture of Adenocarcinoma and Macrophage Cells Within the Fibers

MDA-MB-231 human adenocarcinoma cells (ATCC) and RAW 264.7 mouse macrophage cells (ATCC) were cultured in Dulbecco's modified Eagle's medium (DMEM, Fisher) supplemented with 10% fetal bovine serum (Life Technologies) and 1% penicillin/streptomycin (p/s), media changed every 2 days and passaged at ˜80% confluency using 0.05% Trypsin:EDTA (Life Technologies). CellTracker Green CM FDA dye and CellTracker CM-Dil dye (Life Technologies) were used to label RAW and 231 cells respectively according to manufacturer's instructions. Labeled 231 cells were dispersed in the alginate solution at a concentration of 5×10⁶ cells/mL. Labeled RAW cells were dispersed in CaCl₂ solution at a concentration of 4.5×10⁷ cells/mL. After fiber generation, fibers were cut into approximately 20 mm sections and stored in 24-well cell culture plates containing media and pharmacological drugs. For pharmacological inhibition studies, Gefitinib (G-4408, LC Labs) was used at 10, 50, and 100 nM, Zoledronic acid (Cayman Chemical) at 10, 50, 100 μM, and Rac1 Inhibitor II (CAS 1090893-12-1, Calbiochem) at 10, 50, 100 μM concentrations. A vehicle control of 2% DMSO in cell culture media was also used. The cells were incubated at 37° C., 5% CO2 environment, with media changes every 2 days.

### Example 2

### Covalently-Conjugating Alginate Fibers

Sodium alginate (71238 Sigma, 1.5 g) was dissolved overnight stirring in 150 mL of PBS at room temperature. EDC (E1769 Sigma, 0.597 g) and sulfo-NHS (56485 Sigma, 0.418 g) were added and stirred for 5 minutes, followed by addition of YIGSR (T7154 Sigma) and stirred for 24 at RT under nitrogen. The solution was dialyzed in Millipore-filtered water for 5 days and lyophilized for 8 days. Conjugation was calculated to be 7% from 1H NMR in D2O (FIG. 4). To image the fiber patterns in the absence of cellular additives, the 406 mg sections of sodium alginate were added to a solution of EDC (10.5 mg), sulfo-NHS (4.83 mg), in 7 mL of PBS and stirred for 5 minutes at room temperature. Then fluoresceinamine (201626 Sigma, 1.4 mg) was added and stirred for 24 hours. The fibers were then washed three times with PBS and fluorescently imaged. Peak a correlates with the 4 backbone protons from YIGSR and peak b correlates with the manuronic and gluronic acid ring protons. Approximately 7% conjugation of peptide to the alginate backbone was calculated.

### Example 3

### Concentric Glass Capillary Microfluidic Device Manufacture

Glass capillary tubes from Vitrocom were purchased with inner diameters of 100 μm, 700 μm, and 2000 μm. They were glued in a concentric pattern with Loctite 5 Minute Epoxy and washed several times with water and isopropanol. See, e.g., FIG. 2a.

### Example 4

### Producing 1D, 2D, 3D Architectures

A 3.2% weight solution of sodium alginate was left to gently stir at 3° C. for 5 days in PBS for the second fluid, a 45 mg/mL solution of CaCl₂ in media was prepared for the inner (first) fluid, and a saturated solution of CaSO₄ in PBS for the outer (third) fluid. The solutions were extruded from Harvard Apparatus PhD 2000 syringe pumps and collected in a bath of inner (first) fluid solution without cells.

### Example 5

### Environmental Scanning Electron Microscopy of Alginate Fibers

After fiber production in the absence of cellular additives, the sections of hydrogel were cut into 20 mm sections and submerged in liquid nitrogen for 10 minutes, fractured, and then immediately lyophilized in a LABCONCO Freezone 4.5 Liter Freeze Dry system for 36 hours. The fiber sections were then sputter-coated with ˜80 nm of Au/Pd for imaging.

### Example 6

### Cell Count and Viability Assay

Cell viability was measured every day for 7 days. A 20 mm section of cell fiber was collected in a centrifuge tube and suspended in 2 mL of 0.5M ETA solution for 30 minutes at 37° C. to dissolve the alginate followed by the addition of 100 μL 0.05% Trypsin and incubation at 37° C. for an additional 5 minutes to form single cell suspension. The solution was centrifuged for 5 min at 300 rcf and the resulting cell pellet was re-suspended in 1 mL of fresh cell culture media. A 1:1 mixture of cell suspension and 0.4% Trypan blue solution (Life Technologies) was prepared and counted with a hemocytometer to determine the number of live (unstained) compared to dead (blue stained) cells. 3 counts were averaged for each day.

### Example 7

### Cellular Migration and Correlation Analysis

Confocal image stacks of fiber samples were opened in ImageJ Fiji using the Coloc2 plugin with threshold values consistently set throughout samples for the green (macrophage channels) and the orange (231 cell channels). The outputs of the plugin are displayed as Pearson Correlation Factors above the threshold values, and the 2-D pixel intensity correlation plot.

### Example 8

### Immunocytochemistry of Co-Cultured Fibers

After 4 days in culture, sectioned cell fibers were fixed in 4% paraformaldehyde for 20 minutes and permeabilized with 0.1% Trition X-100 for 30 minutes before blocking with 1% bovine serum albumin (BSA, Sigma) for 1 hour. Primary antibody labeling was performed in 1% BSA in PBS overnight at 4° C. with mouse anti-cortactin (abcam ab33333, 1:500 dilution), or mouse anti-CD44 (abcam ab6124, 1:500 dilution). Secondary antibody labeling was performed in 5% goat serum containing 1% BSA with Alexa Fluor 647-conjugated anti-mouse IgG antibody (Life Technologies A-21236, 1:500 dilution) and DAPI (1:5000 dilution) for 1 hour at room temperature. Immunofluorescent images were taken on a Zeiss 710 multiphoton confocal microscope.

### Example 9

Extruded alginate fibers fastened vertically in a Perkin Elmer 7e dynamic mechanical analyzer (DMA) were measured for frequency sweeps of storage and loss modulus at room temperature. The samples were approximately 3 cm with 0.5 cm of clamping distance. Results are illustrated in FIG. 5.

### Example 10

The ratio of macrophages to tumor cells varies over time in a multilayer fiber system according to the principles of the present disclosure (the method for which is summarized in Example 6 and FIG. 9). To count cells, macrophages (FIG. 9a) and breast cancer (FIG. 9b) cells are split into separate channels and thresholded to highlight cells (FIG. 9c, FIG. 9d). Because the counting is done in a 3D stack, we add the volume of all the macrophage and cancer cells respectively and divide to calculate the RAW/231 ratio.

Unless otherwise explained, all technical and scientific terms used herein have the same meaning as commonly understood by one of ordinary skill in the art to which a disclosed disclosure belongs. The singular terms “a,” “an,” and “the” include plural referents unless context clearly indicates otherwise. Similarly, the word “or” is intended to include “and” unless the context clearly indicates otherwise. “Comprising” means “including”; hence, “comprising A or B” means “including A” or “including B” or “including A and B.” All references cited herein are incorporated by reference.

The disclosure may be further understood by the following non-limiting examples. All references cited herein are hereby incorporated by reference to the extent not inconsistent with the disclosure herewith. Although the description herein contains many specificities, these should not be construed as limiting the scope of the disclosure but as merely providing illustrations of some of the presently preferred embodiments of the disclosure. For example, thus the scope of the disclosure should be determined by the appended aspects and their equivalents, rather than by the examples given.

While the present disclosure can take many different forms, for the purpose of promoting an understanding of the principles of the disclosure, reference will now be made to the embodiments illustrated in the drawings and specific language will be used to describe the same. It will nevertheless be understood that no limitation of the scope of the disclosure is thereby intended. Any alterations and further modifications of the described embodiments, and any further applications of the principles of the disclosure as described herein are contemplated as would normally occur to one skilled in the art to which the disclosure relates.

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure. Thus, it should be understood that although the present disclosure has been specifically disclosed by preferred embodiments, exemplary embodiments and optional features, modification and variation of the concepts herein disclosed may be resorted to by those skilled in the art, and that such modifications and variations are considered to be within the scope of this disclosure as defined by the appended aspects. The specific embodiments provided herein are examples of useful embodiments of the present disclosure and it will be apparent to one skilled in the art that the present disclosure may be carried out using a large number of variations of the devices, device components, methods steps set forth in the present description. As will be obvious to one of skill in the art, methods and devices useful for the present methods can include a large number of optional composition and processing elements and steps.

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

Every formulation or combination of components described or exemplified herein can be used to practice the disclosure, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the aspects herein.

All patents and publications mentioned in the specification are indicative of the levels of skill of those skilled in the art to which the disclosure pertains. References cited herein are incorporated by reference herein in their entirety to indicate the state of the art as of their publication or filing date and it is intended that this information can be employed herein, if needed, to exclude specific embodiments that are in the prior art. For example, when compositions of matter are disclosed, it should be understood that compounds known and available in the art prior to Applicant's disclosure, including compounds for which an enabling disclosure is provided in the references cited herein, are not intended to be included in the composition of matter aspects herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the disclosure without resort to undue experimentation. All art-known functional equivalents, of any such materials and methods are intended to be included in this disclosure. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this disclosure as defined by the appended aspects.

Although the present disclosure has been described with reference to certain embodiments thereof, other embodiments are possible without departing from the present disclosure. The spirit and scope of the appended aspects should not be limited, therefore, to the description of the preferred embodiments contained herein. All embodiments that come within the meaning of the aspects, either literally or by equivalence, are intended to be embraced therein. Furthermore, the advantages described above are not necessarily the only advantages of the disclosure, and it is not necessarily expected that all of the described advantages will be achieved with every embodiment of the disclosure.

1. A method of making a multilayer fiber for use as a biological tissue model, the method comprising:
injecting a first fluid into a first flow channel at a first flow rate;
injecting a second fluid into a second flow channel at a second flow rate, the second flow channel radially surrounding the first flow channel; and
injecting a third fluid into a third flow channel at a third flow rate, the third flow channel radially surrounding the second flow channel and having an outlet downstream of the first and second flow channels; and
extruding a multilayer fiber from the outlet, the multilayer fiber having an outer layer comprising a hydrogel formed by gelation of the second fluid and an inner layer comprising the first fluid.

2. The method of claim 1, wherein the first flow channel is positioned substantially concentrically within the second flow channel.

3. The method of any of claim 1, wherein the second flow channel is positioned substantially concentrically within the third flow channel.

4. The method of claim 1, wherein the second fluid is a viscoelastic material.

5. The method of claim 1, wherein the second fluid comprises alginate.

6. The method of claim 1, wherein the first fluid comprises a tissue culture medium.

7. The method of claim 1, wherein at least one of the first fluid and the third fluid comprises a first gelating agent.

8. (canceled)

9. (canceled)

10. (canceled)

11. The method of claim 1, wherein the multilayer fiber has a folded structure, a first portion of the outer layer of the tubular fiber being in contact with and overlying a second portion of the outer layer of the tubular fiber.

12. (canceled)

13. The method of claim 1, wherein at least one of the first fluid and the second fluid comprises at least one type of cell.

14. (canceled)

15. (canceled)

16. (canceled)

17. The method of claim 1, wherein the second flow rate is at least ten times greater than the first flow rate.

18. (canceled)

19. The method of claim 1, wherein third flow rate is between about one milliliter per hour and about 200 milliliters per hour.

20. The method of claim 1, further comprising segmenting the multilayer fiber into a plurality of multilayer fiber segments.

21. (canceled)

22. A biological tissue model comprising:
a multilayer fiber comprising:
an inner layer comprising a nonsolid medium, and
an outer layer surrounding the inner layer, the outer layer comprising a hydrogel.

23. The biological tissue model of claim 22, wherein the fiber has a structure which is selected from substantially straight and curved.

24. (canceled)

25. The biological tissue model of claim 22, wherein the fiber has a folded structure.

26. The biological tissue model of claim 22, wherein at least one of the outer layer and the inner layer comprises at least one cell type.

27. (canceled)

28. The biological tissue model of claim 22, wherein the hydrogel comprises alginate.

29. The biological tissue model of claim 22, wherein the hydrogel further comprises a linker molecule.

30. The biological tissue model of claim 22, wherein the linker molecule comprises a peptide.

31. The biological tissue model of claim 22, wherein the fiber is formed by: injecting a first fluid into a first flow channel at a first flow rate;
injecting a second fluid into a second flow channel at a second flow rate, the second flow channel radially surrounding the first flow channel; and
injecting a third fluid into a third flow channel at a third flow rate, the third flow channel radially surrounding the second flow channel and having an outlet downstream of the first and second flow channels; and
extruding the fiber from the outlet, the fiber having an outer layer comprising a hydrogel formed by gelation of the second fluid and an inner layer comprising the first fluid.

32. (canceled)
